(19) United States
(12) Patent Application Publication
(10) Pub. No.: US 2025/0099736 A1
(43) Pub. Date: Mar. 27, 2025

(54) CIRCULATION SUPPORT DEVICES, SYSTEMS, AND METHODS

(71) Applicant: Boston Scientific Scimed, Inc., Maple Grove, MN (US)

(72) Inventor: Darrin Dale Beekman, Osseo, MN (US)

(73) Assignee: Boston Scientific Scimed, Inc., Maple Grove, MN (US)

(21) Appl. No.: 18/897,235

(22) Filed: Sep. 26, 2024

Related U.S. Application Data
(60) Provisional application No. 63/540,916, filed on Sep. 27, 2023.

(51) Int. Cl. A61M 60/117; A61M 60/216; A61M 60/411

(52) U.S. Cl. CPC A61M 60/117; A61M 60/216; A61M 60/411

(57) ABSTRACT

A mechanical circulatory support system may include a blood pump, an elongate tube coupled with the blood pump and extending proximally from the blood pump, and a flexible elongate shaft configured to be removably positioned within the elongate tube. The blood pump may be configured to pump blood from a ventricle of a heart of a patient to vasculature of the patient. The elongate shaft may be inserted into the elongate tube prior to or during delivery of the blood pump to the heart to achieve a desired pushability along the elongate tube. The elongate shaft may be removed from the elongate tube to increase a flexibility along the elongate tube to mitigate movement at a proximal end of the elongate tube being transferred to the blood pump positioned in the heart.

### CROSS-REFERENCE TO RELATED APPLICATIONS

This application claims the benefit of priority under 35 U.S.C. § 119 of U.S. Provisional Application No. 63/540,916, filed Sep. 27, 2023, the entire disclosure of which is hereby incorporated by reference.

### TECHNICAL FIELD

The present disclosure pertains to mechanical circulatory support devices. More specifically, the present disclosure relates to devices, systems, and methods of and/or for the delivery of percutaneous ventricular assist devices (PVADs).

### BACKGROUND

A wide variety of intracorporeal and extracorporeal medical devices and systems have been developed for medical use, for example, in cardiac procedures and/or for cardiac treatments. Some of these devices and systems include guidewires, catheters, catheter systems, pump devices, cardiac assist devices, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### BRIEF SUMMARY

This disclosure provides design, material, manufacturing method, and use alternatives for medical devices, including ventricular assist devices.

A first example of a mechanical circulatory support system may comprise a blood pump configured to pump blood from a ventricle of a heart of a patient to vasculature of the patient, an elongate tube coupled with the blood pump and extending proximally from the blood pump, and a flexible elongate shaft configured to be removably positioned within the elongate tube.

Alternatively or additionally to any of the examples above, in another example, a rigidity of the flexible elongate shaft may be greater than a rigidity of the elongate tube.

Alternatively or additionally to any of the examples above, in another example, a rigidity of the flexible elongate shaft may be less than a rigidity of the elongate tube.

Alternatively or additionally to any of the examples above, in another example, the blood pump may further comprise a motor and a motor cable extending proximally from the motor, and wherein the elongate tube may include a lumen and the motor cable extends through the lumen.

Alternatively or additionally to any of the examples above, in another example, the lumen may be configured to receive the motor cable and the flexible elongate shaft.

Alternatively or additionally to any of the examples above, in another example, the lumen may be a first lumen and the motor cable extends through the first lumen, and the elongate tube may include a second lumen extending along the first lumen and configured to removably receive the flexible elongate shaft.

Alternatively or additionally to any of the examples above, in another example, the second lumen may have an interior surface configured to facilitate inserting and removing the flexible elongate shaft into and from the second lumen.

Alternatively or additionally to any of the examples above, in another example, the interior surface may be formed from a polytetrafluoroethylene (PTFE) material.

Alternatively or additionally to any of the examples above, in another example, the elongate tube may comprise a bend at the location proximate the distal end of the elongate tube and the flexible elongate shaft may be configured to extend through the location proximate the distal end and straighten the bend.

In another example, a mechanical circulatory support delivery system may comprise a blood pump, an elongate tube coupled with the blood pump and extending proximally from the blood pump, wherein the elongate tube comprises a lumen through which the motor cable extends, a handle coupled with the elongate tube and configured to receive the elongate motor cable, and a flexible elongate shaft configured to be removably positioned within the elongate tube at a location proximate the handle. The blood pump may include a motor, a motor cable in communication with the motor and extending proximally from the motor, and an impeller in communication with the motor.

Alternatively or additionally to any of the examples above, in another example, the elongate tube may include a side port at a location distal of the handle and the side port is configured to removably receive the flexible elongate shaft.

Alternatively or additionally to any of the examples above, in another example, the handle may include a side port in communication with the elongate tube and the side port is configured to removably receive the flexible elongate shaft.

Alternatively or additionally to any of the examples above, in another example, the lumen may be configured to receive the motor cable and the flexible elongate shaft.

Alternatively or additionally to any of the examples above, in another example, the lumen may be a first lumen and the elongate tube may include a second lumen configured to receive the flexible elongate shaft.

Alternatively or additionally to any of the examples above, in another example, the elongate tube may comprise a bend at a location proximate a distal end of the elongate tube and the flexible elongate shaft may be configured to extend through the location proximate the distal end and straighten the bend.

In another example, a method may comprise inserting a mechanical circulatory support system into a vasculature of a patient, delivering the blood pump through the vasculature of the patient to a heart of the patient, and withdrawing the flexible elongate shaft from the elongate tube. The mechanical circulatory support system may include a blood pump, an elongate tube coupled with the blood pump and extending proximally from the blood pump, and a flexible elongate shaft removably positioned within the elongate tube.

Alternatively or additionally to any of the examples above, in another example, the withdrawing the flexible elongate shaft from within the elongate tube may occur after the blood pump is delivered to the heart of the patient.

Alternatively or additionally to any of the examples above, in another example, the method may further include inserting the flexible elongate shaft into the elongate tube, wherein the elongate tube includes a bend and the flexible elongate shaft is inserted into the elongate tube to at least a location of the bend such that the elongate tube straightens and withdrawing the flexible elongate shaft from the elongate tube includes withdrawing a distal end of the flexible elongate shaft to at least a location proximal of the bend to allow the elongate tube to re-form a shape of the bend prior to delivering the blood pump to the heart of the patient.

Alternatively or additionally to any of the examples above, in another example, the method may further include withdrawing a distal end of flexible elongate shaft from the elongate tube and re-inserting the flexible elongate shaft into the elongate tube.

Alternatively or additionally to any of the examples above, in another example, the flexible elongate shaft may be a first flexible elongate shaft and the method may further comprise withdrawing a distal end of first flexible elongate shaft from the elongate tube and inserting a second flexible elongate shaft into the elongate tube, wherein the second flexible elongate shaft has a rigidity greater than the first flexible elongate shaft.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a schematic perspective view of an illustrative circulatory support system;

FIG. 2 is a schematic partial cross-section of anatomy and a schematic side view of an illustrative percutaneous ventricular assist device (PVAD) within the anatomy;

FIG. 3 is a schematic cross-sectional view of an illustrative PVAD;

FIG. 4 is a schematic diagram of an illustrative circulatory support system, with an elongate shaft spaced from an elongate tube;

FIG. 5 is a schematic diagram of an illustrative circulatory support system of FIG. 4, with the elongate shaft inserted into the elongate tube;

FIG. 6 is a schematic diagram of an illustrative circulatory support system, with a handle portion in cross-section;

FIG. 7 is a schematic diagram of a cross-sectional view of a portion of an illustrative circulatory support system inserted into a vessel of a patient;

FIG. 8 is a schematic cross-sectional view taken at an axial location along an elongate tube of an illustrative circulatory support system, with an elongate shaft inserted into the elongate tube;

FIG. 9 is a schematic cross-sectional view taken at an axial location along an elongate tube of an illustrative circulatory support system, with an elongate shaft inserted into the elongate tube; and

FIG. 10 is a schematic diagram of an illustrative method of using the circulatory support system.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

A variety of circulatory assist or support devices are known for assisting or replacing a pumping function of a heart in a patient with severe heart failure and/or other cardiac conditions. Circulatory support devices may be configured to treat patients with cardiogenic shock, myocardial infarction, acutely decompensated heart failure, and/or other heart related conditions. Additionally or alternatively circulatory assist devices may support a patient during percutaneous coronary interventions and/or other procedures.

Example cardiac circulatory support devices include, but are not limited to, ventricular assist devices (VADs), total artificial hearts, intra-aortic balloon pumps (IABP), and extracorporeal membrane oxygenation (ECMO) devices. Example VADs include left ventricular assist devices (LVADs), right ventricular assist devices (RVADs), and biventricular assist devices (BiVADs). A further illustrative VAD is a percutaneous ventricular assist device (PVAD), which may be inserted into a ventricle (e.g., a left ventricle or a right ventricle) of a heart of a patient via delivery through a femoral artery or vein and/or other suitable vasculature to the ventricle. A PVAD may be placed at a desired location of anatomy of a patient via percutaneous access and delivery, which may enable the PVAD to be used in emergency medicine, a cath lab, and/or other surgical and/or non-surgical settings.

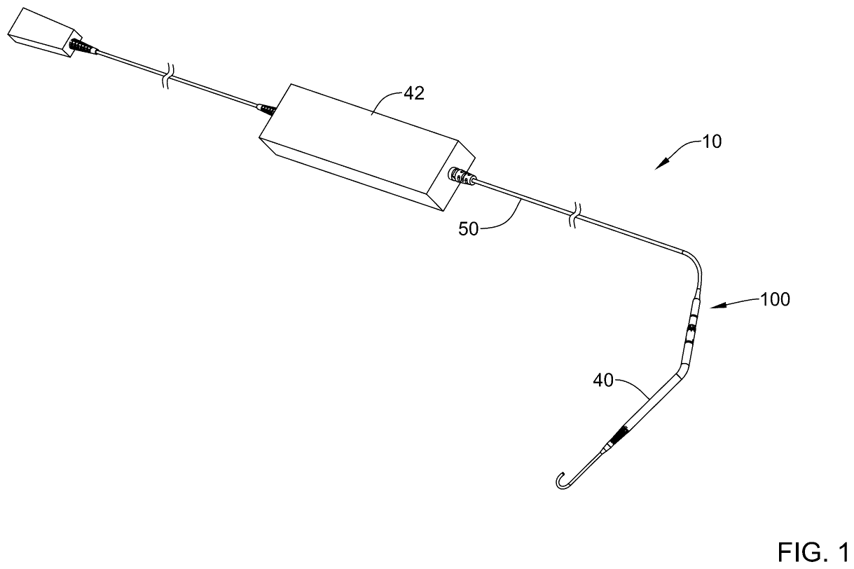

FIG. 1 depicts a schematic view of an illustrative circulatory support system 10. The system 10 may include a percutaneous support device (e.g., a PVAD, such as a blood pump 100), a cannula 40, and an introducer sheath (not shown). In some cases, the system 10 may include a guidewire, but this is not required. In some examples, the introducer sheath, when included, may facilitate percutaneous delivery of the blood pump 100 and the cannula 40 to a target location within a patient (e.g., a target location within a heart of a patient). When positioned at a target site in a heart of a patient, the blood pump 100 may be configured to pump blood from a ventricle of the heart to vasculature of the patient.

The system 10 may further include a proximal housing 42 coupled with (e.g., connected to) an elongate tube 50 (e.g., a catheter and/or other suitable elongate tube), where the elongate tube 50 may be coupled with the blood pump 100 (e.g., a distal end of the elongate tube 50 may be coupled with a proximal end of the blood pump 100). In some examples, at least the proximal housing 42, the elongate tube 50, and/or other components of the system 10 may be configured for delivering the blood pump 100 and the cannula 40 to the target location or site within the patient. In some examples, the guidewire, when included, may also be used to facilitate the delivery of the blood pump 100 and/or the cannula 40.

Although not shown, the system 10 may additionally or alternatively include one or both of a starter tube and a starter tube flushing line. The starter tube, when included, may couple with a cannula delivery tool (e.g., a delivery sheath) which may be configured to receive the cannula 40 prior to and during delivery of the blood pump 100 and the cannula 40 to a target site.

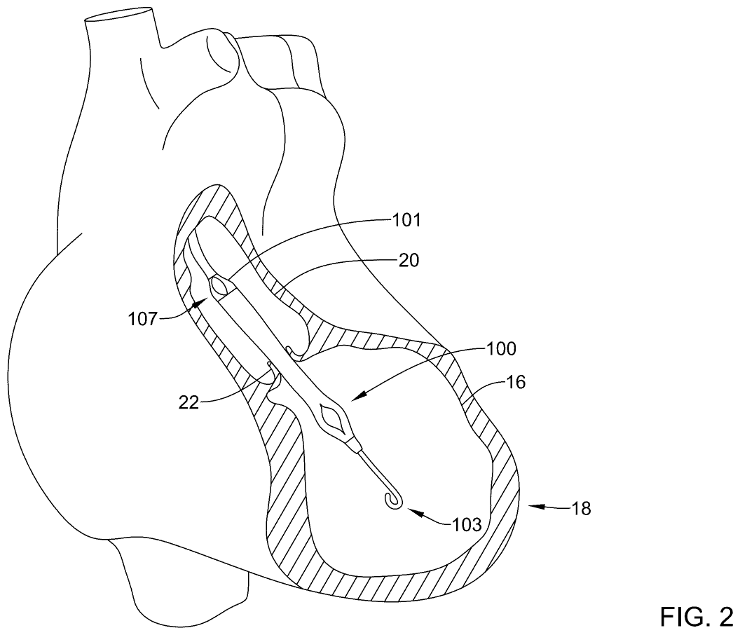

FIG. 2 depicts an illustrative positioning of the blood pump 100 (e.g., a percutaneous circulatory support device, such as a PVAD in an LVAD configuration, etc.) in anatomy of a patient. In FIG. 2, the blood pump 100 is positioned with a distal end 103 located in a left ventricle 16 of a heart 18 and a proximal end 107 in an aorta 20, such that the blood pump 100 extends across an aortic valve 22 between the left ventricle 16 and the aorta 20. With the blood pump 100 extending from the left ventricle 16 to the aorta 20, the blood pump 100 may be configured to pump blood from the left ventricle 16 into the aorta 20 to assist or support blood flow circulation. Other suitable positions of the blood pump relative to the anatomy are contemplated and include, but are not limited to, the distal end 103 of the blood pump being positioned in a right ventricle of the heart 18 with a proximal end being positioned in a pulmonary artery.

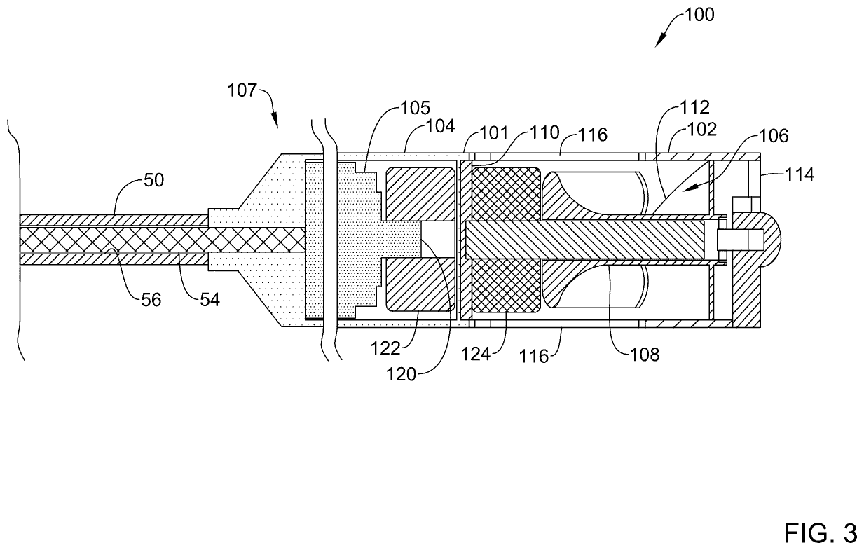

FIG. 3 depicts a schematic cross-sectional view of an illustrative configuration of the blood pump 100. The blood pump 100 may form part of the circulatory support system 10, as discussed, together with a guidewire, an introducer sheath, a controller, a user interface, one or more sensors, and/or other suitable components.

The blood pump 100 may include a housing 101 having an impeller housing 102 and a motor housing 104. The impeller housing 102 and the motor housing 104 may be integrally or monolithically constructed, but this is not required, and the impeller housing 102 and the motor housing 104 may be separate components configured to be removably or permanently coupled. In some configurations, the blood pump 100 may lack a motor housing 104 separate from the impeller housing 102, and the impeller housing 102 may be coupled directly to a motor 105, or the motor housing 104 may be integrally constructed with the motor 105.

The impeller housing 102 may house an impeller assembly 106 and a driven magnet 124, which may be part of or separate from the impeller assembly 106. The impeller assembly 106 may include an impeller shaft 108 that is rotatably supported by at least one bearing, such as a bearing 110 and/or other suitable bearings. The impeller assembly 106 may further include an impeller 112 that rotates relative to the impeller housing 102 to drive blood through the blood pump 100. In some configurations and, for example as illustrated, the impeller shaft 108 and the impeller 112 may be separate components, and in other configurations the impeller shaft 108 and the impeller 112 may be integrated. The impeller assembly 106, as a whole, may be considered a driven component and/or the rotating components of the impeller assembly 106 (e.g., the impeller shaft 108 and/or the impeller 112) may individually or in combination be driven components.

The impeller 112 may be configured within the impeller housing 102 such that as the impeller 112 rotates, blood flows from a blood inlet 114 formed on or at the impeller housing 102, through the impeller housing 102, and out of a blood outlet 116 formed on or at the impeller housing 102. In some configurations, the impeller housing 102 may couple to or include a distally extending cannula (not shown), and the cannula may receive and deliver blood to the inlet 114 (e.g., from the left ventricle 16 of the heart 18 and/or from other suitable locations).

The inlet 114 and the outlet 116 may each have any suitable number of apertures configured to facilitate receiving blood at the blood pump 100 and outputting blood from the blood pump 100, respectively. In some examples, the inlet 114 and/or the outlet 116 may each include multiple apertures and in other examples, one or both of the inlet 114 and the outlet 116 may each include a single aperture.

The inlet and the outlet 116 may each be formed at any suitable location along the impeller housing 102 or other suitable location along the blood pump 100. In some examples, and as depicted in FIG. 2, the inlet 114 may be formed on an end portion (e.g., a distal end portion) of the impeller housing 102 and the outlet 116 may be formed on a side portion (e.g., proximal of the location of the inlet 114) of the impeller housing 102. Other suitable positioning configurations of the inlet 114 and/or the outlet 116 on the impeller housing 102 are contemplated.

The motor housing 104 may house the motor 105, along with other suitable components. In some examples, and as depicted in FIG. 2, the motor housing 104 may house at least the motor 105, the drive shaft 120, and a driving magnet 122.

The motor 105 may be any suitable type of motor. In one example, the motor 105 may be a brushless direct current (DC) motor (BLDC), but other suitable motor types are contemplated.

In operation, the motor 105 may be configured to rotatably drive the impeller 112 relative to the impeller housing 102. In some example configurations, the motor 105 may rotate a drive shaft 120, which is coupled to a driving magnet 122. Rotation of the driving magnet 122 may cause rotation of the driven magnet 124, which is part of or connected to and rotates together with the impeller assembly 106. That is, when the impeller shaft 108 is included in the impeller assembly 106, the impeller shaft 108 and the impeller 112 are configured to rotate with the driven magnet 124. Additionally or alternatively, the motor 105 may couple to the impeller assembly 106 via other suitable components.

A controller (not shown in FIG. 3) may be operably coupled to the motor 105 and configured to control the motor 105 via one or more command signals sent from the controller to the motor 105. The controller may be disposed within the motor housing 104 and/or the controller may be disposed outside of the motor housing 104 (e.g., in a housing of the blood pump 100 independent from the motor housing 104, exterior of the patient, in a housing of the circulatory support system 10 separate from the blood pump 100, etc.). In some embodiments, the controller may include multiple components, one or more of which may be disposed within and/or separately from the motor housing 104.

The motor housing 104 may couple with an elongate tube 50 (e.g., a catheter and/or other suitable elongate tube) at a location of the motor housing 104 opposite the impeller housing 102. The elongate tube 50 may couple to the motor housing 104 in various manners, such as laser welding, soldering, or the like. The elongate tube 50 may extend proximally away from the motor housing 104.

The elongate tube 50 may include one or more lumens for receiving one or more components of a circulation support system including the blood pump 100. In some cases, the elongate tube 50 may be configured to receive and/or carry a motor cable 54 (e.g., one or more cables configured to facilitate operation of the motor 105). In some examples, the elongate tube 50 may receive and/or carry the motor cable 54 within a lumen 56 thereof, and the motor cable 54 may operably couple the motor 105 to the controller (not shown) and/or an external power source (not shown).

The elongate tube 50 may carry one or more transmission cables from one or more sensors proximate the blood pump to a controller. In some cases, one or more of the sensors may be configured to sense a pressure in or around the blood pump 100.

Delivery of blood pumps 100 and/or other suitable circulatory support devices to a target location can be difficult. Circulatory support devices (e.g., the blood pumps 100) are often inserted into the patient through a femoral artery of the patient. When the patient has a tortuous femoral artery, delivery of the circulatory support devices to the heart of the patient can be particularly difficult due to a relatively large diameter and rigid length of the circulatory support device. As such, the elongate tube 50 extending proximal from the blood pump may be required to provide large amounts of push force on the blood pump 100 to facilitate advancing the blood pump 100 through the femoral artery and/or other suitable vessels to the heart of the patient.

It may be advantageous to have a small diameter elongate tube 50 extending proximally from the blood pump 100 to facilitate “dual access” through a sheath inserted into skin and a vessel (e.g., femoral artery or other suitable vessel) of the patient. “Dual access” may refer to inserting two or more catheters or other suitable devices through a sheath (e.g., an introducer sheath, delivery sheath, etc.) at the same time. In some examples, a first catheter may be an elongate tube 50 of the circulatory support system 10 and a second catheter may be used for a percutaneous coronary intervention (PCI), but this is not required.

Further, it may be advantageous to have small diameter elongate tubes 50 to facilitate use of a small diameter repositioning sheath, which may replace the introducer sheath at a femoral artery insertion location after the blood pump 100 has been initially positioned at the target site (e.g., within and/or proximate the heart of the patient). Smaller diameter repositioning sheaths may facilitate improved circulation or perfusion in the vessel of the patient over larger diameter repositioning sheaths by taking up less space in the vasculature of the patient.

Although utilizing small diameter and stiff elongate tubes 50 may be an option to address the need for column strength, or rigidity, or stiffness in the elongate tube 50 (e.g., a longitudinal rigidity or stiffness) to advance the blood pump 100 to the target site while providing space for dual access and/or allowing use of a smaller diameter repositioning sheath, the stiffer the elongate tube 50 is, the more difficult it is to maintain a position of a blood pump 100 at a target site. The difficulty in maintaining a position of the blood pump 100 at the target site may be due to the stiff elongate tube 50 more readily (e.g., more efficiently) transferring patient movement to the blood pump 100 relative to less stiff elongate tubes 50. Additionally, patient safety is a concern when utilizing a stiff elongate tube 50 due to the stiff elongate tube applying larger forces than less stiff elongate tubes 50 to a patient over long dwell times.

As such, an elongate tube that is stiff (e.g., that is rigid and/or includes high column strength) and/or that is kink resistant may be desirable during delivery, but detrimental during device dwell (e.g., dwell may be a time period during which the blood pump 100 is positioned at a target site and the elongate tube 50 extends from the blood pump 100, through the vasculature of the patient, and out of the patient). The concepts discussed herein address a desire to have stiffer or rigid elongate tube 50 during delivery, but a flexible or resilient elongate tube 50 with a smaller diameter during dwell periods.

In one example configuration, the circulatory support system 10 may include an elongate shaft (e.g., a flexible elongate shaft) that may be insertable into the elongate tube 50 to achieve a desired column strength or rigidity or stiffness (e.g., pushability) along the elongate tube 50 and facilitate advancing the blood pump 100 to a target site within the patient. When an elongate shaft insertable into the elongate tube 50 is utilized, prior to or once the blood pump 100 is positioned at the target site, the elongate shaft may be removed from the elongate tube 50 allowing the elongate tube 50, which may be flexible and/or at least partially radially collapsible, to be configured with a small diameter and remain in and extending out of the patient after delivering the blood pump to the heart of the patient. Such a small diameter and flexible and/or at least partially radially collapsible elongate tube 50 may mitigate movement of the patient from being transferred to the blood pump 100 positioned at the target site.

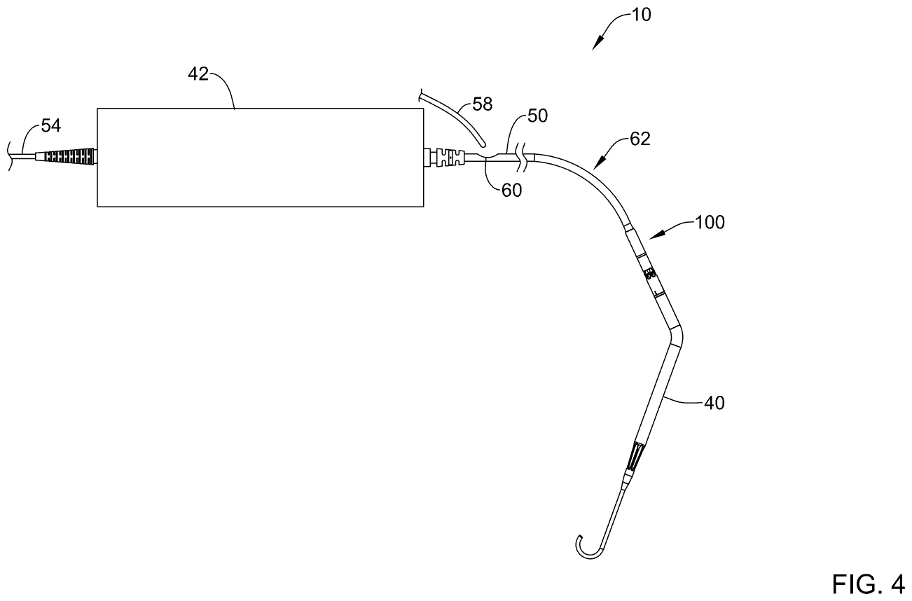

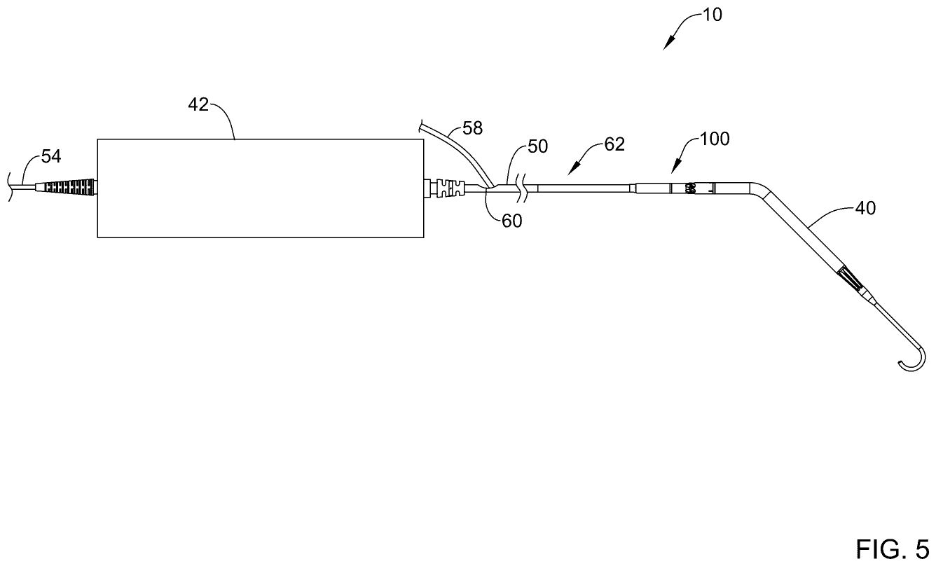

FIG. 4 and FIG. 5 depict schematic side views of an illustrative configuration of the circulatory support system 10. As depicted, the circulatory support system 10 may include, among additional and/or alternative components, the blood pump 100, the cannula 40, the proximal housing 42, and the elongate tube 50 extending proximally from the blood pump 100 to the proximal housing 42. Further, the system 10 may include an elongate shaft 58 and a port 60 configured to be in communication with a lumen of the elongate tube 50, where the elongate shaft 58 may be removably positioned within the elongate tube 50 via the port 60. When inserted in the elongate tube 50, the elongate shaft 58 may increase a rigidity or column strength of the circulatory support system along the elongate tube 50 and as a result, increase the pushability of the system along the elongate tube 50.

The elongate shaft 58 may have any suitable column strength or rigidity or stiffness along its length. An example suitable column strength for the elongate shaft may be in a range of three to fifteen pounds, but this is not required. An example suitable bending stiffness of the elongate shaft may be in a range of 0.4 to 1.5 pound-feet-inches-squared. In some cases, the elongate shaft 58 may have a column strength or rigidity or stiffness that varies along its length and/or circumference. In some examples, the column strength or rigidity or stiffness may be varied along the length and/or circumference of the elongate shaft 58 by changing an outer diameter of the elongate shaft 58, applying a pattern to the elongate shaft 58 (e.g., via laser cutting and/or other suitable technique), using different materials along a length of the elongate shaft 58, and/or varied in one or more other suitable manners.

FIG. 4 depicts the circulatory support system 10 with the elongate tube 50 having a pre-formed bend at a location 62 proximate a distal end of the elongate tube 50 (e.g., just proximal blood pump 100). As depicted in FIG. 4, the elongate shaft 58 has not been inserted into the elongate tube 50, but rather has a distal end aligned with and spaced from a port 60 (e.g., a side port in the elongate tube 50) configured to removably receive the flexible elongate shaft 58.

The pre-formed bend, when included, may facilitate positioning and maintaining the blood pump 100 at a target site. For example, the pre-formed bend may be configured to bend the elongate shaft 58 around an aortic arch or other portion of the patient's vasculature, which may facilitate positioning the blood pump at the target site. Additionally or alternatively, the pre-formed bend in the elongate tube 50 may facilitate decoupling forces acting on the proximal end of the elongate tube 50 and/or proximal portion of the circulatory support system 10 when the blood pump 100 is positioned at a target site in the heart of the patient to better maintain position stability.

FIG. 5 depicts the circulatory support system 10 with the elongate shaft 58 inserted through the port 60 and into the elongate shaft 58. In some examples and as depicted in FIG. 5, the elongate shaft 58 may be inserted to and/or past the location 62 at which the preformed bend is positioned in order to straighten the elongate tube 50. The straightening of the elongate tube 50 may facilitate the system 10 traversing vasculature of the patient. Removing the elongate shaft 58 from a position extending across the location 62 may allow the bend to automatically return (e.g., as the elongate tube 50 may be biased to a bend configuration at the location 62) to the elongate tube 50 and facilitate positioning and maintaining the blood pump 100 in the heart of the patient at a target site.

Straightening the elongate tube 50 may include straightening the elongate tube 50 from a resting radius of curvature until an axis of the elongate tube 50 at the location 62 of the pre-formed bend is aligned with and/or parallel to a central axis of the elongate tube 50 proximal to the location 62 at which the bend is located and/or a central axis of the elongate tube 50 distal of the location 62 at which the bend is located. Alternatively or additionally, straightening the elongate tube 50 may include at least somewhat straightening the bend from the resting radius of curvature to a smaller bend angle relative to the central axis of the elongate tube 50 proximal of the location 62.

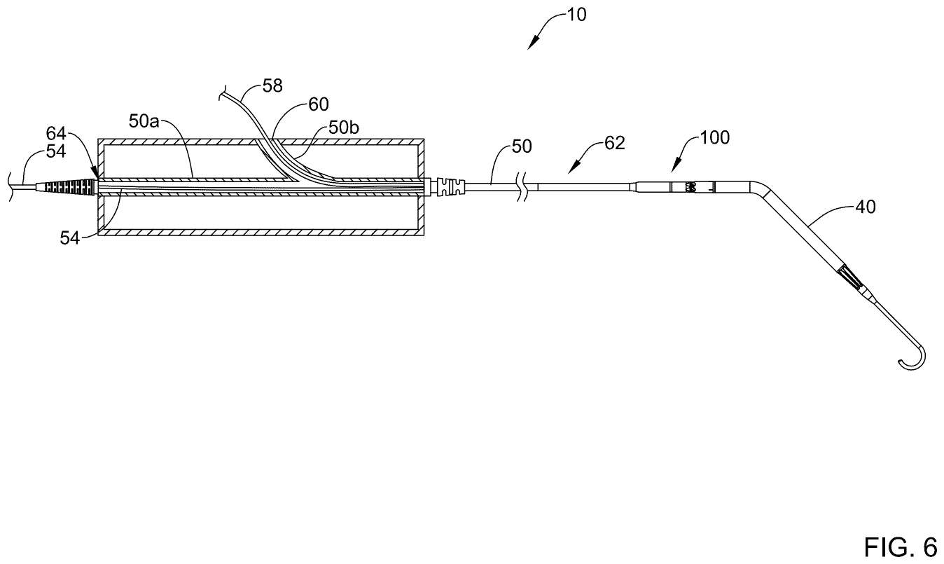

FIGS. 4 and 5 depict the port 60 as a side port at a proximal end of the elongate tube 50 at a location proximate a distal end of the proximal housing 42, but this configuration is not required. For example, the port 60 may be an end port and/or a port located at one or more other suitable locations along the elongate tube 50 and/or other suitable components of the circulatory support system 10. In some examples, the port 60 may be located in the proximal housing 42 of the system 10, as depicted in FIG. 6.

When the port 60 is in the proximal housing 42, the port 60 may be positioned at any suitable location along the proximal housing 42. In some examples, the port 60 may be located on a side of the proximal housing 42 (e.g., as a side port as depicted in FIG. 6) or a proximal end of the proximal housing 42, as desired.

When the port 60 is included in the proximal housing 42, the elongate tube 50 may include a bifurcation at the proximal end thereof (e.g., within the proximal housing 42 and/or at any other suitable location). For example, a first portion 50a of elongate tube 50 at the bifurcation may extend to and/or be in communication with a motor cable port 64 along the proximal housing 42 and a second portion 50b of the elongate tube 50 at the bifurcation may extend to and/or be in communication with the port 60. Alternatively, the motor cable 54 may exit the elongate tube 50 through a side port within the proximal housing 42 and a proximal terminal end of the elongate tube 50 may be in communication with the port 60. Other suitable configurations of the elongate tube 50 and the port 60 are contemplated to facilitate inserting the elongate shaft 58 into the elongate tube 50 and/or removing the elongate shaft 58 from the elongate tube 50.

The elongate shaft 58 may be any suitable type of shaft 58. For example, the elongate shaft 58 may be a mandrel, a tube, a wire, and/or other suitable elongate shaft configuration.

The elongate shaft 58 may be formed from any suitable material. The material may be selected based on one or more considerations including, but not limited to, kink resistance, lubricity, flexibility, rigidity or stiffness, resilience, and/or other suitable considerations. Example suitable materials include, but are not limited to, metals, polymers, nickel-titanium alloys (e.g., NITINOL and/or other suitable nickel-titanium alloys), stainless steel, polytetrafluoroethylene (PTFE), polyamid (e.g., VESTAMID and/or other suitable polyamid material) and/or other suitable materials. In some examples, the elongate shaft 58 may be at least partially formed from a nickel-titanium alloy to mitigate the chances of the elongate shaft kinking and/or other suitable material that reduces a likelihood of the elongate shaft 58 kinking as it traverses through the elongate tube 50 and/or within vasculature. In some examples, the elongate shaft 58 may include a metal core (e.g., a stainless steel core) coated with PTFE and/or other materials that facilitates sliding the elongate shaft 58 within the elongate tube 50.

The elongate shaft 58 may have any suitable length. For example, the elongate shaft 58 may have a length equal to or greater than a length of the elongate tube 50, but this is not required. In some examples, the elongate shaft 58 may have a length at least as long as a distance from the port 60 to the location 62 at which the pre-formed bend is located in the elongate tube 50 (e.g., when the pre-formed bend is located in the elongate tube 50) such that the elongate shaft 58 may cause the elongate tube 50 to straighten at the location 62 relative to a resting position of the elongate tube 50 at the location 62 of the pre-formed bend.

The elongate shaft 58 may have any suitable outer diameter. In some examples, a size of the outer diameter of the elongate shaft 58 may be determined based on an inner diameter of the elongate tube 50, an outer diameter of the motor cable(s) 54, a needed stiffness along the elongate tube 50 for delivery the blood pump 100 to the target site (e.g., where a larger diameter elongate shaft 58 is generally stiffer than a smaller diameter elongate shaft 58 of the same material), and/or one or more other suitable considerations.

Any suitable combination of characteristics (e.g., material, diameter, stiffness, etc.) may be utilized in the elongate shaft 58. In some cases, a user (e.g., physician, clinician, etc.) may have two or more elongate shafts 58 and may select an initial shaft based on an expected tortuosity of the vasculature of the patient. During the procedure and/or at other times, an initially selected elongate shaft 58 may be switched out for a different elongate shaft 58 having one or more characteristics different than the initial elongate shaft 58. In some examples, two or more elongate shafts 58 may be utilized simultaneously. In some examples, the elongate shafts 58 may be selected based on a needed stiffness or rigidity along a length of the elongate tube 50 to facilitate traversing the vasculature of the patient.

The elongate tube 50 may have any suitable configuration and may be formed from one or more layers. One or more of the layers may be formed through an extrusion process, a bending process, and/or other suitable processes. In some examples, the elongate tube 50 may have an outer polymer layer (e.g., a polyamid layer), an inner polymer layer, and a stainless steel braided layer sandwiched between the inner polymer layer and the outer polymer layer. In some cases, the outer polymer layer may be reflowed onto the braided layer, but this is not required. In some examples, the elongate tube 50 may be a single layer. The elongate tube 50 may be radially flexible and/or collapsible, but this is not required.

The inner layer or at least an inner surface of the elongate tube 50 may be formed from a lubricious material. In one example, the inner surface of the elongate tube 50 may be or may be formed from PTFE material to facilitate slidably receiving the elongate shaft 58.

The elongate shaft 58 may be flexible so as to be able to traverse a tortuous vasculature, but be stiff enough to add a sufficient stiffness or rigidity to the elongate tube 50 to facilitate advancing the blood pump 100 through the tortuous vasculature to the heart of the patient. In some examples, the elongate shaft 58 may be flexible and stiffer than the elongate tube 50 (e.g., the elongate shaft 58 may be more rigid than or have a greater rigidity than the elongate tube 50) to provide a desired amount of pushability along the elongate tube 50 and/or cause the elongate tube 50 to straighten at the location 62 of the pre-formed bend. Alternatively, in some examples the elongate shaft 58 may be flexible and less stiff than the elongate tube 50 (e.g., the elongate shaft 58 may have a rigidity that is less than a rigidity of the elongate tube 50), but still stiff enough to provide the desired amount of pushability along the elongate tube 50 when inserted through the elongate tube.

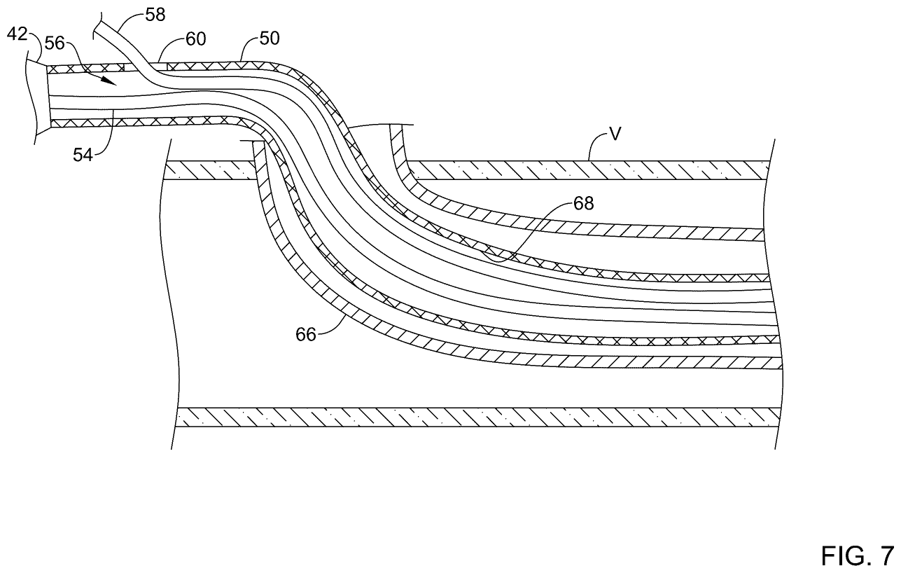

FIG. 7 depicts a schematic cross-sectional view of a blood vessel with a sheath 66 inserted at least partially into the blood vessel V. The elongate tube 50 may extend within and/or through the sheath 66 and into the blood vessel V. The elongate tube 50 may include the motor cable 54 and the elongate shaft 58 within a lumen 56 thereof. In some examples, an inner surface 68 of the elongate tube 50 may be formed from and/or include a lubricious material (e.g., PTFE, etc.) to facilitate slidably receiving the elongate shaft 58. The elongate shaft 58 may extending proximally out of the port 60 and may be configured to be slidably adjustable relative to the elongate tube 50 to increase or decrease a rigidity along the elongate tube 50, as discussed herein.

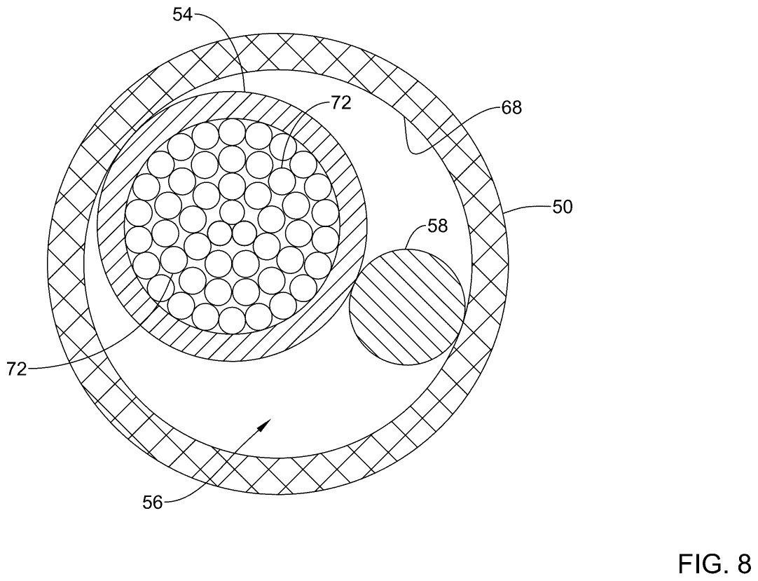

FIG. 8 depicts a schematic cross-sectional view taken at an axial location along an illustrative configuration of the elongate tube 50 with the elongate shaft 58 and the motor cable 54 inserted in the lumen 56 thereof, where the elongate tube 50 has a single lumen (e.g., the lumen 56). Although only a single motor cable 54 is depicted within the lumen 56, one or more motor cables 54 and/or other cables (e.g., sensor cables, pressure sensor cables, fiber optic cables, etc.) extending proximally from blood pump 100 may extend within the lumen 56. The motor cable(s) 54 may include one or more wires 72, as desired. Further, although only a single elongate shaft 58 is depicted within the lumen 56, one or more elongate shafts 58 may be utilized to achieve a desired rigidity along a length of the elongate tube 50.

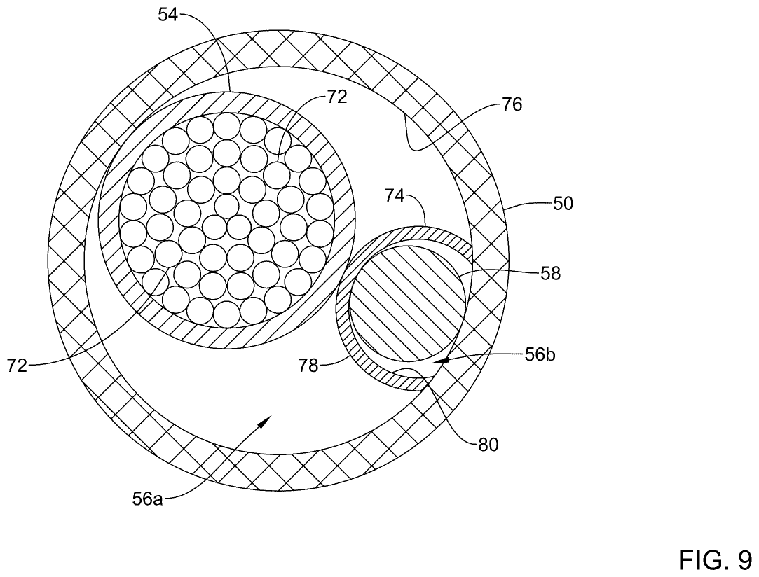

FIG. 9 depicts a schematic cross-sectional view taken at an axial location along an illustrative configuration of the elongate tube 50 with the elongate shaft 58 and the motor cable 54 inserted therein, where the elongate tube 50 has a first lumen 56a and a second lumen 56b. In some examples, the elongate tube 50 may at least partially define the first lumen 56a and a separator 74 (e.g., a tube, a wall, etc.) may at least partially define the second lumen 56b. Although two lumens are in the configuration of the elongate tube 50 depicted in FIG. 9, a single lumen or more than two lumens may be utilized.

In some configurations and as depicted in FIG. 9, the first lumen 56a may be defined by an interior surface 76 of the elongate tube 50 and a first surface 78 of the separator 74 (e.g., an outer surface when the separator 74 is a tube) having a second surface 80 (e.g., an inner surface when the separator 74 is a tube) defining the second lumen 56b. The motor cable(s) 54 may be received in and/or extend through the first lumen 56a. In some cases, one of the elongate tube 50 and the separator 74 (when in a tube configuration) may be considered a first tube and the other of the elongate tube 50 and the separator 74 may be considered a second tube.

In some cases, the separator 74 may individually be a full tube where the second surface 80 defines an entire circumference of the second lumen 56b. Alternatively or additionally, the separator 74 may form a tube with the elongate tube 56, where the second surface 80 of the separator 74 defines a portion of a circumference of the second lumen 56b and the inner surface 76 of the elongate tube 50 defines a portion of the second lumen 56b. Although the separator 74 is depicted as having a round cross-section in FIG. 9, this is not required and the cross-section may take on one or more other suitable shapes. In some cases, the elongate shaft 58 may be removably received in and/or removably extend through the second lumen 56b.

The first lumen 56a and the second lumen 56b may be formed in any suitable manner. In some examples, the first lumen 56a and the second lumen 56b may be a double lumen extrusion, may be formed from a co-extrusion, may be separately extruded, and/or may be formed and/or coupled to one another in any suitable manner. In some examples, the first lumen 56a may be defined by an inner surface of a first tube, the second lumen 56b may be defined by an inner surface of a second tube, and a third tube may extend around an outer surface of the first tube and the second tube, where the third tube may define an outer surface of the elongate tube 50. Alternatively, in some examples the first lumen 56a and the second lumen 56b may be within a single tube and separated by a wall of the single tube. Other suitable configurations of the elongate tube 50 with or without one or more other sub-tubes are contemplated.

The separator 74 may be formed from any suitable material. As depicted in FIG. 9, the separator 74 may be formed from a different material than the material of the elongate shaft 58, but this is not required and the separator 74 may be formed from the same material(s) used to form the elongate tube 50 and/or may be monolithic with elongate tube. Similar to other surfaces that may contact the elongate shaft 58, the inner surface 80 defining the second lumen 56b may be configured to facilitate inserting and removing the flexible elongate shaft into and/or from the second lumen 56b. For example, the inner surface 80 may be formed from or with a lubricious material or other suitable material, such as PTFE and/or other materials suitable for facilitating slidably receiving the elongate shaft 58. However, in some cases, the inner surface 80 may be formed from a material other than a lubricious material.

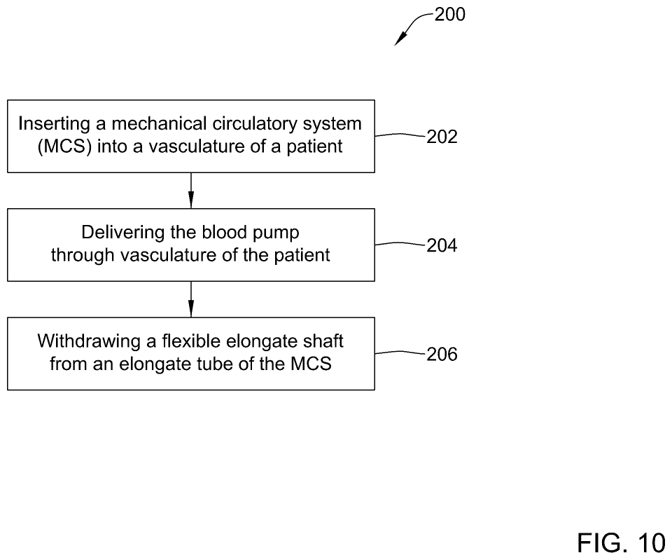

FIG. 10 schematically depicts a diagram of an illustrative method or technique 200. The method 200 may be configured to facilitate delivering and/or positioning a blood pump at a target site in a heart of a patient and maintaining a position of the blood pump at the target site. The blood pump may have a configuration of the blood pumps described herein and/or one or more other suitable blood pump configurations.

The method 200 may include inserting 202 a mechanical circulatory system (MCS) in a vasculature of a patient. The MCS may have any suitable configuration including one or more of the configurations described herein and/or otherwise. In some examples, the MCS may include a blood pump, an elongate tube coupled with the blood pump and extending proximally from the blood pump, and an elongate shaft, where the elongate shaft may be removably positioned within the elongate tube. Illustratively, the elongate shaft may be selectively utilized to adjust a stiffness or rigidity along a length of the elongate shaft to facilitate delivery of the blood pump to a target site and/or facilitate maintaining the blood pump at the target site.

Prior to, during, or after initial insertion of the MCS into the vasculature of the patient, a user (e.g., physician, clinician, etc.) may select an elongate shaft (e.g., a flexible elongate shaft) that may have a stiffness or rigidity that will achieve a desired pushability along a length of the elongate tube as the blood pump traverses the vasculature on its way to a heart of the patient. Once the elongate shaft is or the elongate shafts are selected, the elongate shaft(s) may be inserted into the elongate tube. In some examples, the elongate shaft may be selected and inserted into the elongate tube prior to initial insertion of a MCS device (e.g., blood pump) into the vasculature of the patient. In some cases, MCS device pack may come with the elongate shaft pre-loaded into the elongate tube.

The elongate shaft may be inserted into the elongate tube any desired distance. In some cases, where the elongate tube includes a bend at one or more locations along the length of the elongate shaft (e.g., proximate a distal end of the elongate shaft adjacent the blood pump and/or at one or more other suitable locations), the elongate shaft may be inserted to a depth of at least one of the bends to straighten the elongate tube at the location(s) of the bend(s) and increase pushability of the MCS through the vasculature of the patient.

Once the MCS has been inserted into the vasculature of the patient, the blood pump may be delivered 204 through the vasculature of the patient to a heart of the patient (e.g., a target site within the heart of the patient). In some cases, as the blood pump is being delivered, if the user feels resistance and is having difficulty passing a particularly tortuous portion of the vasculature due to bending along the elongate tube as result of a push force on the elongate tube, the user may slide or otherwise withdraw the elongate shaft out of the elongate tube without withdrawing the elongate tube from the patient and insert a stiffer elongate shaft into elongate shaft and proceed with the delivery of the blood pump.

As discussed, the elongate shaft may be withdrawn 206 from the elongate tube at any suitable time before, during, or after delivery of the blood pump to the target site at the pump. Further, withdrawing 206 the elongate shaft from the elongate tube may include entirely withdrawing the shaft and/or partially withdrawing the elongate shaft from the elongate tube.

In some cases, as the blood pump is approaching the aortic arch, a user may at least partially withdraw the elongate shaft from the elongate tube such that a distal terminal end of the elongated tube is proximal of a location of the pre-formed bend in the elongate tube. Withdrawing the elongate shaft to at least this portion may allow the elongate tube to automatically re-form a shape of the bend to facilitate passing the blood pump through the aortic arch and into the heart of the patient.

Although not required, once the blood pump is delivered to the target site at the heart of the patient, the elongate shaft may be fully withdrawn from the elongate tube (e.g., a distal end of the elongate shaft be positioned outside of the elongate tube). Fully withdrawing the elongate shaft from the elongate tube may facilitate having a desired amount of stiffness or flexibility along the elongate tube during a dwell time or period to mitigate a likelihood of movement at a proximal end of the elongate tube causing movement of the blood pump.

If the blood pump is inadvertently adjusted during a dwell period and/or the blood pump needs to be adjusted for one or more other reasons, a user may be able to re-insert the elongate shaft or a different elongate shaft (e.g., a second elongate shaft) to facilitate adjusting a location of the blood pump. In some cases, as the blood pump is already at the heart, a less stiff elongate shaft may be re-inserted (e.g., re-inserted, as in an elongate shaft, even if the shafts are different, is being re-inserted) into the elongate tube to provide sufficient stiffness along the elongate tube for a user to transfer movement at a proximal end of the MCS to the blood pump and position the blood pump at the target site or a new target site. Further, in some cases, the elongate shaft may be re-inserted into the elongate tube to facilitate withdrawing the blood pump from the patient and/or for other suitable reasons. The ability to re-insert the elongate shaft may be beneficial when the elongate tube has lost stiffness from being in the patient due to fluid absorption by polymers of the elongate tube and/or may be beneficial for other reasons.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A mechanical circulatory support system comprising:
a blood pump configured to pump blood from a ventricle of a heart of a patient to vasculature of the patient;
an elongate tube coupled with the blood pump and extending proximally from the blood pump; and
a flexible elongate shaft configured to be removably positioned within the elongate tube.

2. The system of claim 1, wherein a rigidity of the flexible elongate shaft is greater than a rigidity of the elongate tube.

3. The system of claim 1, wherein a rigidity of the flexible elongate shaft varies along a length of the flexible elongate shaft.

4. The system of claim 1, wherein:
the blood pump further comprises:
a motor; and
a motor cable extending proximally from the motor, and
wherein the elongate tube includes a lumen and the motor cable extends through the lumen.

5. The system of claim 4, wherein the lumen is configured to receive the motor cable and the flexible elongate shaft.

6. The system of claim 4, wherein:
the lumen is a first lumen and the motor cable extends through the first lumen, and
the elongate tube includes a second lumen extending along the first lumen and configured to removably receive the flexible elongate shaft.

7. The system of claim 6, wherein the second lumen has an interior surface configured to facilitate inserting and removing the flexible elongate shaft into and from the second lumen.

8. The system of claim 7, wherein the interior surface is formed from a polytetrafluoroethylene (PTFE) material.

9. The system of claim 1, wherein the elongate tube comprises a bend at a location proximate a distal end of the elongate tube and the flexible elongate shaft is configured to extend through the location proximate the distal end and straighten the bend.

10. A mechanical circulatory support delivery system comprising:
a blood pump comprising:
a motor;
a motor cable in communication with the motor and extending proximally from the motor; and
an impeller in communication with the motor;
an elongate tube coupled with the blood pump and extending proximally from the blood pump, wherein the elongate tube comprises a lumen through which the motor cable extends;
a housing coupled with the elongate tube and configured to receive the elongate motor cable; and
a flexible elongate shaft configured to be removably positioned within the elongate tube at a location proximate the housing.

11. The system of claim 10, wherein the elongate tube includes a side port at a location distal of the housing and the side port is configured to removably receive the flexible elongate shaft.

12. The system of claim 10, wherein the housing includes a side port in communication with the elongate tube and the side port is configured to removably receive the flexible elongate shaft.

13. The system of claim 10, wherein the lumen is configured to receive the motor cable and the flexible elongate shaft.

14. The system of claim 10, wherein:
the lumen is a first lumen; and
the elongate tube includes a second lumen configured to receive the flexible elongate shaft.

15. The system of claim 10, wherein the elongate tube comprises a bend at a location proximate a distal end of the elongate tube and the flexible elongate shaft is configured to extend through the location proximate the distal end and straighten the bend.

16. A method, comprising:
inserting a mechanical circulatory support system into a vasculature of a patient, the mechanical circulatory support system comprising:
a blood pump;
an elongate tube coupled with the blood pump and extending proximally from the blood pump; and
a flexible elongate shaft removably positioned within the elongate tube;
delivering the blood pump through the vasculature of the patient to a heart of the patient; and
withdrawing the flexible elongate shaft from the elongate tube.

17. The method of claim 16, wherein the withdrawing the flexible elongate shaft from within the elongate tube occurs after the blood pump is delivered to the heart of the patient.

18. The method of claim 16, further comprising:
inserting the flexible elongate shaft into the elongate tube, wherein the elongate tube includes a bend and the flexible elongate shaft is inserted into the elongate tube to at least a location of the bend such that the elongate tube straightens; and
withdrawing the flexible elongate shaft from the elongate tube includes withdrawing a distal end of the flexible elongate shaft to at least a location proximal of the bend to allow the elongate tube to re-form a shape of the bend prior to delivering the blood pump to the heart of the patient.

19. The method of claim 16, further comprising:
withdrawing a distal end of flexible elongate shaft from the elongate tube; and
re-inserting the flexible elongate shaft into the elongate tube.

20. The method of claim 16, wherein the flexible elongate shaft is a first flexible elongate shaft and the method further comprises:
withdrawing a distal end of first flexible elongate shaft from the elongate tube; and
inserting a second flexible elongate shaft into the elongate tube, wherein the second flexible elongate shaft has a rigidity greater than the first flexible elongate shaft.
